(19) United States
(12) Patent Application Publication
(10) Pub. No.: US 2025/0290090 A1
(43) Pub. Date: Sep. 18, 2025

(54) METHODS FOR INDUCING APOMIXIS IN PLANTS

(71) Applicant: CHINA NATIONAL RICE RESEARCH INSTITUTE, Hangzhou, Zhejiang (CN)

(72) Inventors: Kejian WANG, Hangzhou (CN); Yong HUANG, Hangzhou (CN)

(73) Assignee: CHINA NATIONAL RICE RESEARCH INSTITUTE, Hangzhou, Zhejiang (CN)

(21) Appl. No.: 19/010,241

(22) Filed: Jan. 6, 2025

(30) Foreign Application Priority Data
Mar. 13, 2024 (CN) 202410284881.8

(51) Int. Cl. C12N 15/82

(52) U.S. Cl. CPC C12N 15/8287; C12N 15/8233

(57) ABSTRACT

The present disclosure belongs to the field of plant breeding and biotechnology, disclosing a method for inducing apomixis in plants, specifically involving the application of the WUS gene in inducing apomixis. The inventors' research revealed that the MOC3 gene in rice (renamed as OsWUS) can be used to construct apomictic lines. In these lines, the seed-setting rate and the induction rate of clonal seeds exhibited varying degrees of change, with some lines maintaining a normal seed-setting rate and achieving a clonal seed efficiency of up to 22%. Therefore, the present disclosure has significant application value.

### CROSS-REFERENCE TO RELATED APPLICATION

This application claims priority to Chinese Patent Application No. 202410284881.8, filed on Mar. 13, 2024, the entire contents of each of which are hereby incorporated by reference.

### SEQUENCE LISTING

The instant application contains a Sequence Listing which has been submitted electronically in XML format and is hereby incorporated by reference in its entirety. The XML copy, created on Dec. 19, 2024, is named “2024 Dec. 19-Sequence Listing-6A802-H003US00” and is 32,596 bytes in size.

### TECHNICAL FIELD

The present disclosure generally relates to the field of plant breeding and biotechnology, and in particular, to a method for inducing apomixis in plants, particularly the application of the WUS gene in inducing apomixis.

### BACKGROUND

Heterosis refers to the phenomenon where hybrid offspring surpass their parents in vitality, stress resistance, and yield, which has been widely applied in agricultural production. Currently, hybrid varieties account for approximately 50% of the rice-growing area in China, contributing significantly to national food security. However, due to the inability to inherit heterosis through offspring, hybrid seed production requires substantial annual investment in labor and resources due to genetic recombination and segregation. This cumbersome process not only increases risk and cost but also limits the further promotion of hybrid varieties. Thus, enabling hybrids to self-propagate seeds is considered the ultimate goal of hybrid breeding. Apomixis is a type of asexual reproduction through seeds, producing offspring genetically identical to the mother plant. Introducing apomixis into hybrids allows stable inheritance of heterozygous genotypes, preventing trait segregation, thereby stabilizing heterosis, greatly simplifying the breeding process, reducing risks and costs, and enhancing the utilization of heterosis. In recent decades, significant breakthroughs have been achieved in artificial apomixis technology through elucidating molecular mechanisms of reproductive development and advances in gene editing technology. Multiple research teams have successfully developed clonal seeds in model plants like Arabidopsis and rice using MiMe strategies (producing clonal gametes genetically identical to the mother plant) and haploid induction strategies. Raphaël Mercier's team was the first to attempt combining MiMe (Atspo11-1-Atrec8-Atosd1) with cenh3 chromosome elimination in Arabidopsis, achieving approximately 40% clonal seeds (Marimuthu M P, Jolivet S, Ravi M, Pereira L, Davda J N, Cromer L, Wang L, Nogué F, Chan S W, Siddiqi I, Mercier R. Synthetic clonal reproduction through seeds. Science. 2011 Feb. 18; 331 (6019): 876. doi: 10.1126/science. 1199682.). Notably, despite the highly conserved function of CENH3 across multiple species, no apomixis system based on CENH3 has been reported outside of Arabidopsis. In 2019, Venkatesan Sundaresan's team combined MiMe (pair1-rec8-osd1) with the BBM1 expression component driven by the egg cell-specific promoter pDD45, establishing the first apomixis system in a conventional rice variety, Kitaake, and obtaining clonal seeds of Kitaake. Although the seed-setting rate was low, the efficiency of clonal seeds reached up to 29% (Khanday I, Skinner D, Yang B, Mercier R, Sundaresan V. A male-expressed rice embryogenic trigger redirected for asexual propagation through seeds. Nature. 2019 January; 565 (7737): 91-95. doi: 10.1038/s41586-018-0785-8.). In the same year, Wang Kejian's team used CRISPR/Cas9 technology to develop Fix lines in the indica-japonica hybrid rice Chunyou 84 (CY84), incorporating simultaneous mutations of MiMe (pair1-rec8-osd1) and the mtl gene, marking the first attempt of successfully producing clonal seeds in hybrid rice. The seed-setting rate ranged from 3.7% to 5.2%, and clonal seeds accounted for 4.7% to 9.5% of all viable seeds (Wang C, Liu Q, Shen Y, Hua Y, Wang J, Lin J, Wu M, Sun T, Cheng Z, Mercier R, Wang K. Clonal seeds from hybrid rice by simultaneous genome engineering of meiosis and fertilization genes. Nat Biotechnol. 2019 March; 37 (3): 283-286. doi: 10.1038/s41587-018-0003-0.). Unlike the CENH3 strategy, these two methods do not require a hybridization process and can achieve clonal seeds through self-pollination, opening new research avenues for fixing crop heterosis.]I; In 2022, Emmanuel Guiderdoni and Raphaël Mercier's team integrated BBM1 and MiMe into a single vector in the hybrid rice BRS-CIRAD 302, creating an apomixis system with a clonal seed proportion exceeding 90%, although the seed-setting rate under greenhouse conditions was only about 30% (Vernet A, Meynard D, Lian Q, Mieulet D, Gibert O, Bissah M, Rivallan R, Autran D, Leblanc O, Meunier A C, Frouin J, Taillebois J, Shankle K, Khanday I, Mercier R, Sundaresan V, Guiderdoni E. High-frequency synthetic apomixis in hybrid rice. Nat Commun. 2022 Dec. 27; 13 (1): 7963. doi: 10.1038/s41467-022-35679-3.). In 2023, Wang Kejian's team further explored the potential of BBM4, a homologous gene of BBM1, in apomixis systems. They successfully developed Fix2 lines capable of apomixis in the indica-japonica hybrid rice Chunyou 84 (CY84). These clonal plants were phenotypically similar to the wild-type hybrid rice and maintained a high seed-setting rate of 80.9% to 82.0%, although the proportion of clonal seeds reached only 2.3% in this study (Wei X, Liu C, Chen X, Lu H, Wang J, Yang S, Wang K. Synthetic apomixis with normal hybrid rice seed production. Mol Plant. 2023 Mar. 6; 16 (3): 489-492. doi: 10.1016/j.molp.2023.01.005.). Recent studies indicate that combining dandelion PAR gene-induced parthenogenesis with the MiMe (pair1-rec8-osd1) strategy can achieve apomixis in hybrid rice, maintaining normal seed-setting rates while reaching a maximum clonal seed efficiency of 67.7% (Song M, Wang W, Ji C, Li S, Liu W, Hu X, Feng A, Ruan S, Du S, Wang H, Dai K, Guo L, Qian Q, Si H, Hu X. Simultaneous production of high-frequency synthetic apomixis with high fertility and improved agronomic traits in hybrid rice. Mol Plant. 2024 Jan. 1; 17 (1): 4-7. doi: 10.1016/j.molp.2023.11.007.). These findings not only provide new research pathways for fixing crop heterosis but also hold profound scientific and practical implications for ensuring global food security.

Currently, significant progress has been made in rice apomixis technology, but critical challenges remain. On the one hand, while apomictic plants can maintain normal seed-setting rates, the efficiency of clonal seed production remains relatively low. On the other hand, when the efficiency of clonal seeds approaches 100%, the seed-setting rate of apomictic plants significantly decreases. This demonstrates that an apomixis technology system capable of ensuring 100% clonal efficiency while maintaining normal seed-setting rates has not yet been developed, limiting the practical application of rice apomixis technology. Therefore, it is urgent to explore and identify more genes related to apomixis to further optimize and enhance existing technological systems.

### SUMMARY

The objective of the present disclosure is to prepare apomictic lines. Through research, it was discovered that the WUS gene in rice and other plants can be used to construct apomictic lines, thereby completing the present disclosure.

The present disclosure first provides the application of a WUS gene in preparing apomictic lines, wherein the plant is a monocotyledon or dicotyledon; more, the plant is from the families Poaceae, Fabaceae, or Brassicaceae; most, the plant is rice, maize, millet, wheat, barley, sorghum, soybean, or rapeseed.

Further provided is a binary expression vector for inducing apomictic lines, comprising a WUS gene driven by an egg cell-specific promoter; the WUS gene originates from monocotyledons or dicotyledons; more, the WUS gene originates from plants in the families Poaceae, Fabaceae, or Brassicaceae; most, the WUS gene originates from rice, maize, millet, wheat, barley, sorghum, soybean, or rapeseed. The WUS gene comprises the full-length genomic sequence or full-length coding sequence.

Furthermore, the vector also carries components capable of producing MiMe, wherein MiMe includes simultaneous mutations of PAIR1, REC8, and OSD1. Components capable of producing MiMe include: A DNA sequence encoding PARI1 sgRNA driven by a U3 promoter, a DNA sequence encoding REC8 sgRNA driven by a U3 promoter, and a DNA sequence encoding OSD1 sgRNA driven by a U3 promoter.

For example, in rice, which is a diploid, introducing MiMe components can produce tetraploid offspring, and the WUS expression component can induce haploid production. When MiMe components and WUS expression components are combined, tetraploids can be reduced to diploids, thereby producing clonal seeds. Therefore, MiMe components and the WUS expression components must be integrated into a single vector to induce the formation of apomictic lines.

Specifically, the base vector is pCAMBIA 1300; other vectors include the pCAMBIA series (excluding pCAMBIA1300), pGreen series, pBIN series, pBI series, and pHELLSGATE series. The promoter is an egg cell-specific promoter, selected from AtDD45 (At2g21740), AtEC1.1 (At1g76750), AtEC1.3 (At2g21750), AtEC1.4 (At4g39340), AtEC1.5 (At5g64720), OsECA1 (LOC_Os03g18530), OsECA2 (LOC_Os11g06730), OsECA3 (LOC_Os12g06970).

Preferably, the OsWUS gene in rice comprises the full-length genomic sequence or the full-length coding sequence.

The present disclosure further provides a method for inducing apomixis in plants, which comprises the following steps: Introducing a binary expression vector containing MiMe-producing components and a WUS ectopic expression component driven by an egg cell-specific promoter into the target plant; screening for MiMe and WUS ectopic expression lines in the transgenic plants to obtain apomictic lines.

Specifically, the plant is a monocotyledon or dicotyledon; more, the plant belongs to the families Poaceae, Fabaceae, or Brassicaceae; most, the plant is rice, maize, millet, wheat, barley, sorghum, soybean, or rapeseed.

In a specific implementation, the introduction is performed via a gene gun or Agrobacterium-mediated transformation.

Optionally, the method further includes a step of screening for apomictic plants with normal seed-setting rates and high clonal seed efficiency.

For example, in rice, a diploid plant, introducing MiMe components can produce tetraploid offspring, while ectopic expression of the WUS gene induces haploid production. Thus, combining MiMe components and WUS ectopic expression components can reduce tetraploids to diploids, resulting in clonal seeds. Only by co-expressing MiMe components and WUS ectopic expression components (e.g., integrated into one expression vector) can apomictic lines be successfully induced.

For some plants like maize and millet, MiMe-producing components have not yet been constructed. However, with the maturity of gene-editing systems and genetic transformation technologies in maize and millet, MiMe components for these plants can be developed. Combined with haploid induction by maize and millet WUS genes, it will be possible to create apomictic lines based on the WUS gene in maize and millet.

The WUSCHEL (WUS) gene is considered an important plant stem cell regulatory gene, playing a key role in maintaining shoot apical meristem function. In crops like Arabidopsis and maize, studies have demonstrated that overexpression of the WUS gene can promote somatic embryogenesis and shoot organ regeneration in tissue culture (Lowe K, Wu E, Wang N, Hoerster G, Hastings C, Cho M J, Scelonge C, Lenderts B, Chamberlin M, Cushatt J, Wang L, Ryan L, Khan T, Chow-Yiu J, Hua W, Yu M, Banh J, Bao Z, Brink K, Igo E, Rudrappa B, Shamseer P M, Bruce W, Newman L, Shen B, Zheng P, Bidney D, Falco C, Register J, Zhao Z Y, Xu D, Jones T, Gordon-Kamm W. Morphogenic Regulators Baby boom and Wuschel Improve Monocot Transformation. Plant Cell. 2016 September; 28 (9): 1998-2015. doi: 10.1105/tpc. 16.00124.). Specifically, the WUS gene in Arabidopsis has a rice homolog, MOC3, which encodes a member of the WOX protein family and is involved in the formation and development of rice tiller buds. When this gene is functionally disrupted, the formation of tiller buds is hindered, resulting in a phenotype of reduced tiller number (Tanaka W, Ohmori Y, Ushijima T, Matsusaka H, Matsushita T, Kumamaru T, Kawano S, Hirano H Y. Axillary Meristem Formation in Rice Requires the WUSCHEL Ortholog TILLERS ABSENT1. Plant Cell. 2015 April; 27 (4): 1173-84. doi: 10.1105/tpc. 15.00074.). However, the inventors' research revealed that the MOC3 gene in rice (renamed as OsWUS) can be used to construct apomictic lines. In these lines, the seed-setting rate and the induction rate of clonal seeds exhibited varying degrees of change, with some lines maintaining a normal seed-setting rate and achieving a clonal seed efficiency of up to 22%.

In studies related to rice, reports have shown that apomictic plants based on BBM1 achieved near 100% clonal seed induction efficiency, though the seed-setting rate under greenhouse conditions reached only about 40%. In contrast, apomictic plants based on BBM4 maintained normal seed-setting rates, but the maximum clonal seed induction rate was only 2.3%. Compared to these, the apomictic lines based on OsWUS in the present disclosure not only maintained normal seed-setting rates but also achieved a maximum clonal seed efficiency of 22%, approximately ten times that of BBM4. Additionally, although apomictic plants based on ToPAR achieved normal seed-setting rates and a maximum clonal seed efficiency of 67.7%, the fact that ToPAR is derived from dandelion rather than a native rice gene might limit its practical application in rice apomixis. Therefore, the present disclosure, which utilizes an apomixis system based on OsWUS, demonstrates significant advantages. These advantages include significantly improving clonal seed induction efficiency while maintaining seed-setting rates and using OsWUS, a native rice gene, which is better suited for the application and promotion of rice apomixis technology.

### BRIEF DESCRIPTION OF THE DRAWINGS

The present disclosure is further described in terms of exemplary embodiments. These exemplary embodiments are described in detail with reference to the drawings. It should be noted that the drawings are not to scale. These embodiments are non-limiting exemplary embodiments, in which like reference numerals represent similar structures throughout the several views of the drawings, and wherein:

FIG. 1 is a schematic diagram illustrating the binary vector that combines the MiMe system with ectopic expression of the OsWUS gene according to the present disclosure;

FIG. 2 are schematic diagrams illustrating of showing the positive identification results of transgenic plants according to the present disclosure;

FIG. 3 is a schematic diagram illustrating representation of the genotypes exhibiting triple homozygous mutations in the PAIR1, REC8, and OSD1 genes according to the present disclosure;

FIG. 4 is a schematic diagram illustrating phenotypic analysis comparing the wild-type Chunyou 84 (CY84) with the T225 #6 lines according to the present disclosure;

FIG. 5 is a schematic diagram illustrating comparative phenotypic analysis between ZmWUS haploid plants and Zheng58 diploid plants according to the present disclosure; and

FIG. 6 is a schematic diagram illustrating comparative phenotypic analysis between SiWUS haploid plants and millet Ci846 diploid plants according to the present disclosure.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The present disclosure is further elucidated through specific embodiments but is not limited thereto.

In current research, artificial apomixis technology mainly involves the combined application of the MiMe strategy and haploid induction strategy. First, the genomic sequence of the OsWUS gene in rice was successfully cloned, including its exons and introns, designated as gOsWUS. Subsequently, the knockout components for the three genes PAIR1, REC8, and OSD1, along with the gOsWUS expression element driven by the egg cell-specific promoter pAtDD45 from Arabidopsis, were integrated into the pCAMBIA1300 binary vector, refining the construction of the T225 vector (sgMiMe_pAtDD45: gOsWUS). Through Agrobacterium-mediated transformation, the T225 vector was introduced into the indica-japonica hybrid rice Chunyou 84 (CY84) to create apomictic lines. The objective of the present disclosure is to screen for apomictic plants with normal seed-setting rates and high clonal seed efficiency.

### EXAMPLES

### Example 1: Creation of Apomictic Lines in Rice

### 1. Cloning and Sequence Analysis of the OsWUS Gene in Rice

DNA was extracted from the leaves of Chunyou 84 (CY84) using the CTAB method. Using the primers 56780-cds-F1 and 56780-cds-R1, the genomic sequence of the OsWUS gene was amplified, including three exons and two introns, and designated as gOsWUS.

56780-cds-F1 (SEQ ID No: 4):
ATGGATCACATGCAGCAGCAGCAGCGGCAGCAGGTG
56780-cds-R1(SEQ ID No: 5):
TCACATGGACCCTGCAGGGTAAGGTGAGCATGAG

The sequence of gOsWUS (SEQ ID No: 1, including two intron sequences) is as follows:

ATGGATCACATGCAGCAGCAGCAGCGGCAGCAGGTGGGTGGAGGG
GGAGGAGAGGAGGTGGCGGGGAGGGGTGGTGTGCCGGTGTGCCG
GCCGAGCGGGACGAGGTGGACGCCGACGACGGAGCAGATCAAGAT
CCTGCGGGAGCTGTACTACAGCTGCGGCATCAGGTCGCCCAACTC
GGAGCAGATCCAGCGGATCGCCGCCATGCTGCGCCAGTACGGCCG
CATCGAGGGCAAGAACGTCTTCTACTGGTTCCAGAACCACAAGGC
CCGCGAGCGCCAGAAGAAGCGCCTCACCACGCTCGACGTCACCAC
CACCACCGCCGCCGCCGCCGACGCCGACGCCAGCCACCTCGCCGT
CCTCTCCCTCTCGCCTACAGCAGCTGGTACGTTGCTGCGTCATGG
CTAATTCCGATCGCTGCTTCCCTGCTAAGCTGTAATGCGCGAGCC
GGCGCCGAGCCGCCGATCGATGCTTCTGCGTGTGCAGGCGCGACG
GCTCCCTCTTTCCCGGGCTTCTACGTCGGCAATGGCGGCGCCGTG
CAGACGGATCAGGCCAACGTCGTCAACTGGGACTGCACCGCCATG
GCAGCCGAGAAAACCTTCCTGCAGGTATGATCACACGTACTACTA
CCTCCTCCAGGTGTGTGTGAATTCACCATGCAAGAGCAAGCTAAT
GTGCAATGCTGCAGGACTACATGGGCGTGAGCGGCGGCGGCGGCG
CCGCCGCGGCGGCCCCGACGCCGTGGGCGATGACGACGACGACTC
GCGAGCCCGAGACGCTTCCACTCTTCCCAGTCGTCGGCGGCGGCG
GCGACGGCGCGCATCGTCACGCCGGCCACGGCGGTTTCCCGTCCA
ACTTCCAGCGCTGGGGTTCTGCTGCTGCTACCACCAACACCATTA
CGGTCCAGCAGCATTTGCAGCAGCACAACTTTTACAGCAGCAGCA
GCAGCCAGCTGCACAGCCAGGATGGGCCGGCAGCAGGCACATCCC
TGGAGCTCACTCTCAGCTCCTACTACTGCTCATGCTCACCTTACC
CTGCAGGGTCCATGTGA.

The two introns contained are as follows:

Intron 1 is (SEQ ID No: 2):
GTACGTTGCTGCGTCATGGCTAATTCCGATCGCTGCTTCCCTGCT
AAGCTGTAATGCGCGAGCCGGCGCCGAGCCGCCGATCGATGCTTC
TGCGTGTGCAG.
Intron 2 is (SEQ ID No: 3):
GTATGATCACACGTACTACTACCTCCTCCAGGTGTGTGTGAATTC
ACCATGCAAGAGCAAGCTAATGTGCAATGCTGCAG.

### 2. Construction of sgMiMe Vector

The main steps are as follows (specific operations can be performed with slight modifications based on the method described in Wang C, Shen L, Fu Y, Yan C, Wang K. A Simple CRISPR/Cas9 System for Multiplex Genome Editing in Rice. J Genet Genomics. 2015 Dec. 20; 42 (12): 703-6. doi: 10.1016/j.jgg.2015.09.011):

1) Construction of the sgMiMe Intermediate Vector

The target sequences of the three genes PAIR1, REC8, and OSD1 were referenced from the target sites reported in the paper (Wang C, Liu Q, Shen Y, Hua Y, Wang J, Lin J, Wu M, Sun T, Cheng Z, Mercier R, Wang K. Clonal seeds from hybrid rice by simultaneous genome engineering of meiosis and fertilization genes. Nat Biotechnol. 2019 March; 37 (3): 283-286. doi: 10.1038/s41587-018-0003-0.), with the PAM sequences underlined. The specific sequences are as follows:

PAIR1 Target Site (SEQ ID No: 6):
AAGCAACCCAGTGCACCGCTGG
REC8 Target Site (SEQ ID No: 7):
CGGAGAGCCTTAGTGCCATGGG
OSD1 Target Site (SEQ ID No: 8):
CTGCCGCCGACGAGCAACAAG

Complementary DNA sequences were designed for each of the genes PAIR1, REC8, and OSD1. GGCA was added to the forward target sequence to create the forward primer, and AAAC was added to the reverse complementary target sequence to create the reverse primer.

The SK-gRNA intermediate vector contains two AarI restriction sites. After digestion with AarI, a vector with sticky ends was formed. The forward and reverse primers of the target sequences were annealed to form sticky-ended fragments. These fragments were ligated to the vector using T4 ligase, forming intermediate vectors for single target genes, labeled as SK-gPAIR1, SK-gREC8, and SK-gOSD1.

2) Construction of the sgMiMe Vector

The pCAMBIA1300 binary vector was digested with KpnI and BamHI to obtain the KpnI-BamHI-linearized pCAMBIA1300 vector. SK-gPAIR1, SK-gREC8, and SK-gOSD1 were digested with KpnI+SalI, XholI+NheI, and XbaI+BglII, respectively, to recover the external fragments SK-gPAIR1IKpnI+SalI, SK-gREC8lXhoI+NheI, and SK-gOSD1/XbaI+BglII. Using T4 ligase, the KpnI-BamHI-linearized pCAMBIA1300 vector was ligated with the external fragments SK-gPAIR1IKpnI+SalI, SK-gREC8lXhoI+NheI, and SK-gOSD1lXbal+BglII to construct the sgMiMe vector.

### 3. Construction of T225 (sgMiMe_pAtDD45: gOsWUS) Vector

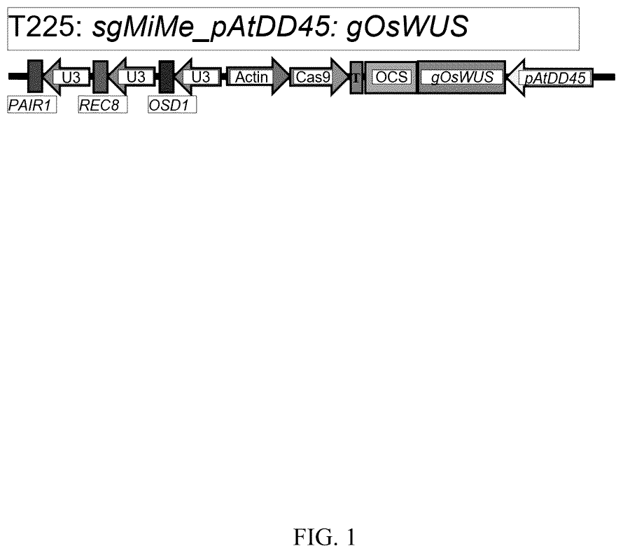

The primer combinations ool-Pmel-F+pAtDD45-overlap-R, pAtDD45-56780-F+56780-overlap-R, and OCS-F+OCS-Pmel-R were used to amplify the egg cell-specific promoter pAtDD45 from Arabidopsis, the genomic sequence of OsWUS (gOsWUS), and the OCS terminator fragment, respectively. The sgMiMe vector was digested with Pmel to obtain the Pmel-linearized sgMiMe vector. Using homologous recombination, pAtDD45, gOsWUS, the OCS terminator fragment, and the Pmel-linearized sgMiMe vector were ligated to complete the construction of the T225 vector (sgMiMe_pAtDD45: gOsWUS) (FIG. 1). Specific primer information is as follows:

ool-Pmel-F (SEQ ID No: 9):
ctgtcaaacactgatagtttAAATGTTCCTCGCTGACGTAAGAAG;
pAtDD45-overlap-R (SEQ ID No: 10):
TATTCTTTCTTTTTGGGGTTTTTG;
pAtDD45-56780-F (SEQ ID No: 11):
AACCCCAAAAAGAAAGAATAATGGATCACATGCAGCAG;
56780-overlap-R (SEQ ID No: 12):
cctgcaggtcgactctagaggatccTCACATGGACCCTGCAGGGT
AAGGTG;
OCS-F (SEQ ID No: 13):
CTCTAGAGTCGACCTGCAGGCATGC;
OCS-Pmel-R (SEQ ID No: 14):
TCGTTTCCCGCCTTCAGTTTTCCCAGTCACGACGTTGTAAAACG.

### 4. Creation of Apomictic Lines in Rice

### 1) Acquisition of T225 Transgenic Plants

The expression vector T225 (sgMiMe_pAtDD45: gOsWUS) was introduced into the Agrobacterium strain EHA105 via electroporation. This binary expression vector was subsequently transferred into the callus of the indica-japonica hybrid rice Chunyou 84 (CY84) through Agrobacterium-mediated genetic transformation. The specific transformation method involved sterilizing the embryos of Chunyou 84 (CY84) hybrid rice seeds and inoculating them onto induction medium. After one week of culture, robust, light yellow, and loose embryogenic calli were selected as transformation recipients. Calli were infected with the EHA 105 strain carrying the T225 (sgMiMe_pAtDD45: gOsWUS) plasmid and cultured in the dark at 25° C. for three days. Resistant calli were screened on selection medium containing 50 mg/L hygromycin and then transferred to differentiation medium, where they were cultured under light at 26° C. Transgenic plants that grew normally on the differentiation medium were selected for rooting culture for 2 weeks, resulting in a total of 29 T225 transgenic plants.

### 2) Genotyping of T225 Transgenic Plants

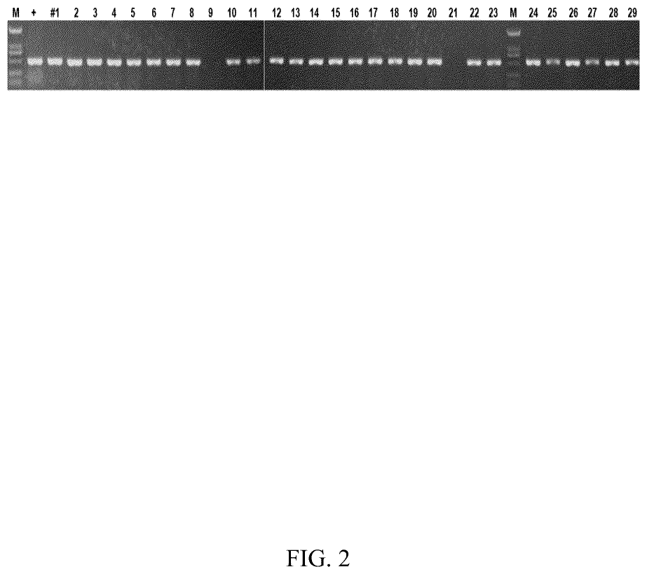

Genomic DNA was extracted from these 29 plants using the CTAB method. Using the primer combination DD45-PCR-F and 56780-PCR-R, transgene-positive identification was conducted on the 29 transgenic lines. It was found that 27 lines contained the pAtDD45: gOsWUS expression component (FIG. 2). The primer sequences used were as follows:

DD45-PCR-F (SEQ ID No: 15):
AGGAGCGCTACTGATTCAACATGCC;
56780-PCR-R (SEQ ID No: 16):
TTCTGGAACCAGTAGAAGACGTTC.

Next, the editing status of the three genes, PAIR1, REC8, and OSD1, was assessed. DNA fragments of the target regions for PAIR1, REC8, and OSD1 were amplified using the respective primer combinations PAIR1-HF1+PAIR1-HB1, REC8-HF1+REC8-HB1, and OSD1-HF2+OSD1-HB2. Genotyping of the amplified DNA fragments was performed using the Hi-TOM technique (Liu Q, Wang C, Jiao X, Zhang H, Song L, Li Y, Gao C, Wang K. Hi-TOM: a platform for high-throughput tracking of mutations induced by CRISPR/Cas systems. Sci China Life Sci. 2019 January; 62 (1): 1-7. doi: 10.1007/s11427-018-9402-9). The primer sequences used were as follows:

PAIR1-HF1 (SEQ ID No: 17):
GGAGTGAGTACGGTGTGCCTTCTTGCGCGCGAGAAGAGTCTC;
PAIR1-HB1 (SEQ ID No: 18):
GAGTTGGATGCTGAGTGGGAGATGTAGTGCGTGGGTCTTG;
REC8-HF1 (SEQ ID No: 19):
GGAGTGAGTACGGTGTGCTTGGGTTAGTGAGGAGAT;
REC8-HB1 (SEQ ID No: 20):
GAGTTGGATGCTGAGTGGTGCGATCGGAACTATGGAGAC;
OSD1-HF2 (SEQ ID No: 21):
GGAGTGAGTACGGTGTGCTATCAGGAGGACGACGTCGCCG;
OSD1-HB2 (SEQ ID No: 22):
GAGTTGGATGCTGAGTGGCTCCTCCTCTTGGGTGTAGC.

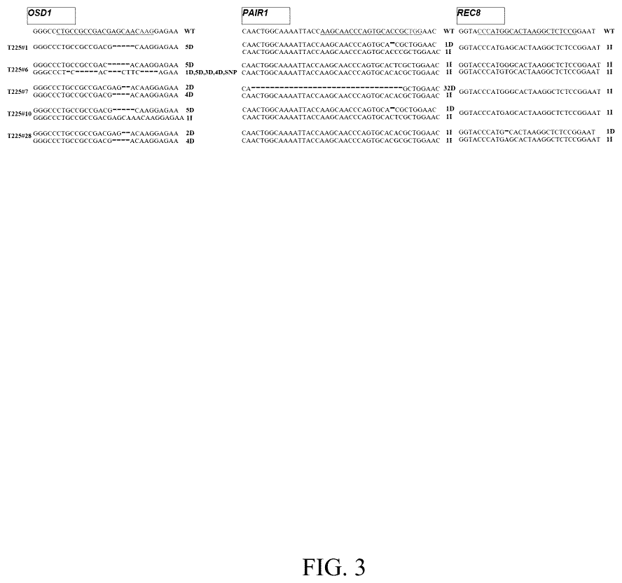

A total of 5 lines (T225 #1, T225 #6, T225 #7, T225 #10, T225 #28) were identified to carry mutations in all three genes, PAIR1, REC8, and OSD1 (FIG. 3). Combining these results with the transgene-positive identification confirmed that these 5 lines contained the pAtDD45: gOsWUS expression component, verifying them as apomictic lines based on OsWUS.

### 3) Phenotypic Identification of T225 Apomictic Lines

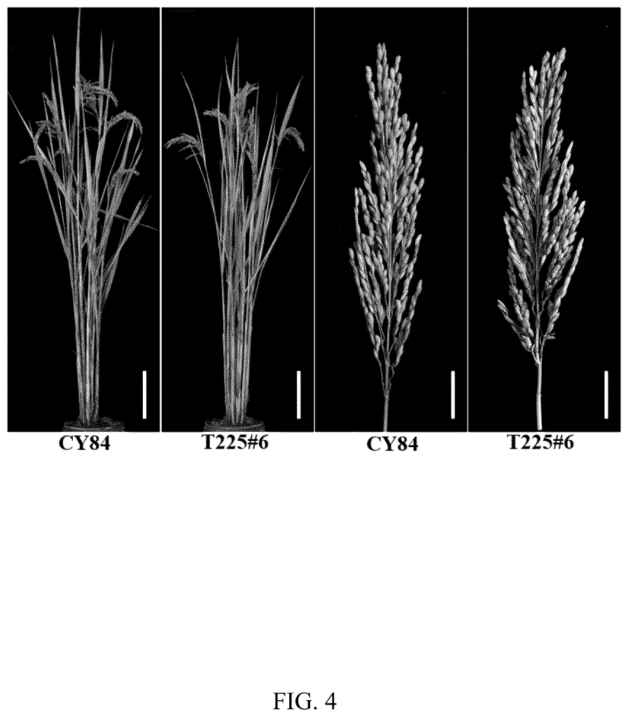

During the vegetative growth stage, these 5 apomictic lines exhibited normal morphology. Surprisingly, at the maturity stage, these 5 apomictic lines showed a high seed-setting rate ranging from 72.0% to 83.3%, comparable to the seed-setting rate of Chunyou 84 (CY84) wild type (80.2+2.3%) (FIG. 4). To verify whether these high seed-setting apomictic plants could produce clonal seeds, the ploidy levels of their T1 progeny were analyzed using flow cytometry. It was found that 4 lines (T225 #1, T225 #6, T225 #7, and T225 #28) produced diploid progeny at a rate of 0.5% to 21.7%, while the remaining line (T225 #10) produced only tetraploids. Notably, the T225 #6 line not only maintained a normal seed-setting rate but also achieved a clonal seed efficiency of 21.7% (Table 1).

TABLE 1
Statistical Analysis of Seed-Setting Rates and
Clonal Seed Efficiency in OsWUS Apomictic Lines

| | | | | | [symbol] Proportion |
|---|---|---|---|---|---|
| | | | | | of Diploids |
| | [symbol] Seed-Setting | [symbol] Number of | [symbol] Number of | [symbol] Number of | (%) |
| Material | Rate | Plants Tested in | Diploid | Tetraploid | [symbol] |
| ID | (%) | T1 Generation | Plants | Plants | (%) |
| CY84 | 80.2 ± 2.3 | / | / | / | / |
| T225#1 | 82.8 ± 3.3 | 191 | 5 | 186 | 2.6 |
| T225#6 | 80.6 ± 3.7 | 92 | 20 | 72 | 21.7 |
| T225#7 | 83.3 ± 1.1 | 192 | 1 | 191 | 0.5 |
| T225#10 | 78.5 ± 3.9 | 96 | 0 | 96 | 0.0 |
| T225#28 | 72.0 ± 2.3 | 192 | 34 | 158 | 17.7 |

### Example 2: Creation of ZmWUS Haploid Induction Lines

### 1. Cloning and Sequencing of the Maize WUS Gene

Following the method in Example 1, the primers ZmWUS-F1 and ZmWUS-R1 were used to amplify Zheng58, obtaining the genomic sequence of the ZmWUS gene (NP_001105961), designated as gZmWUS.

ZmWUS-F1 (SEQ ID No: 23):
ATGGCGGCCAATGCGGGCGGCGGTGG;
ZmWUS-R1 (SEQ ID No: 24):
TCACATGCTCCCTGCAGCAGGGTAAG.

### 2. Construction of the ZmWUS Ectopic Expression Vector

The primer combinations KpnI-F+pAtDD45-overlap-R and pAtDD45-ZmWUS-F+ZmWUS-overlap-R were used to amplify the egg cell-specific expression promoter pAtDD45 from Arabidopsis and the genomic sequence of ZmWUS, which is gZmWUS, respectively. The pCAMBIA1300 vector was digested with KpnI and BamHI to obtain a KpnI-BamHI-linearized vector. The fragments of pAtDD45, gZmWUS, and the linearized vector were ligated using homologous recombination to construct the ZmWUS ectopic expression vector (pAtDD45: gZmWUS). Specific primer information is as follows:

Kpnl-F (SEQ ID No: 25):
tacgaattcgagctcggtacAAATGTTCCTCGCTGACGTAAGAAG;
pAtDD45-overlap-R (SEQ ID No: 26):
TATTCTTTCTTTTTGGGGTTTTTG;
pAtDD45-ZmWUS-F (SEQ ID No: 27):
aaccccaaaaagaaagaataATGGCGGCCAATGCGGGCGGCGGTGG;
ZmWUS-overlap-R (SEQ ID No: 28):
caggtcgactctagaggatcTCACATGCTCCCTGCAGCAGGG.

### 3. Creation of Maize Haploid Induction Lines

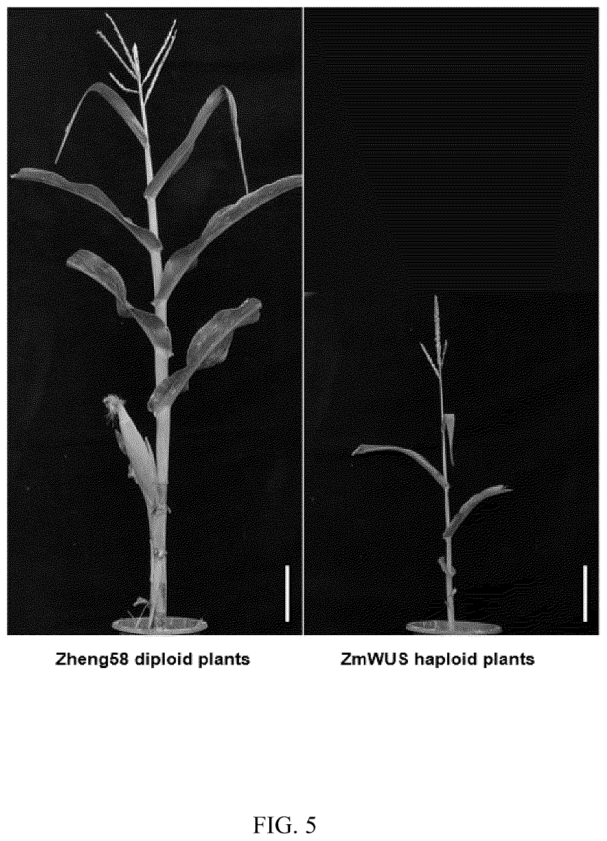

The ZmWUS ectopic expression vector (pAtDD45: gZmWUS) was introduced into Zheng58 following the method in Example 1. A total of 9 ZmWUS ectopic expression plants were obtained. To verify whether the ZmWUS ectopic expression plants could produce haploids, the ploidy levels of their T1 progeny were analyzed using flow cytometry. It was found that the ZmWUS ectopic expression lines produced haploids at a rate of 0.5% to 1.2%. The phenotypes of ZmWUS haploid plants and Zheng58 diploid plants are shown in FIG. 5.

### Example 3: Creation of Millet Haploid Induction Lines

### 1. Cloning and Sequencing of the Millet WUS Gene

Following the method in Example 1, the primers SiWUS-F1 and SiWUS-R1 were used to amplify the millet variety Ci846, obtaining the genomic sequence of the SiWUS gene (XP_022680535), designated as gSiWUS.

SiWUS-F1 (SEQ ID No: 29):
ATGGCGGCCAATGTGGGCGGAAAG
SiWUS-R1 (SEQ ID No: 30):
TCACATGGTCCCTGCAGGGTAAGG.

### 2. Construction of the SiWUS Ectopic Expression Vector

The primer combinations KpnI-F+pAtDD45-overlap-R and pAtDD45-SiWUS-F+SiWUS-overlap-R were used to amplify the egg cell-specific expression promoter pAtDD45 from Arabidopsis and the genomic sequence of SiWUS, which is gSiWUS, respectively. Following the method in Example 2, the pAtDD45, SiWUS fragment, and KpnI-BamHI-linearized vector were ligated to construct the SiWUS ectopic expression vector (pAtDD45: gSiWUS). Specific primer information is as follows:

Kpnl-F (SEQ ID No: 31):
tacgaattcgagctcggtacAAATGTTCCTCGCTGACGTAAGAAG;
pAtDD45-overlap-R (SEQ ID No: 32):
TATTCTTTCTTTTTGGGGTTTTTG;
pAtDD45-SiWUS-F (SEQ ID No: 33):
aaccccaaaaagaaagaataATGGCGGCCAATGTGGGCGGAAAG;
SiWUS-overlap-R (SEQ ID No: 34):
caggtcgactctagaggatcTCACATGGTCCCTGCAGGGTAAGG.

### 3. Creation of Millet Haploid Induction Lines

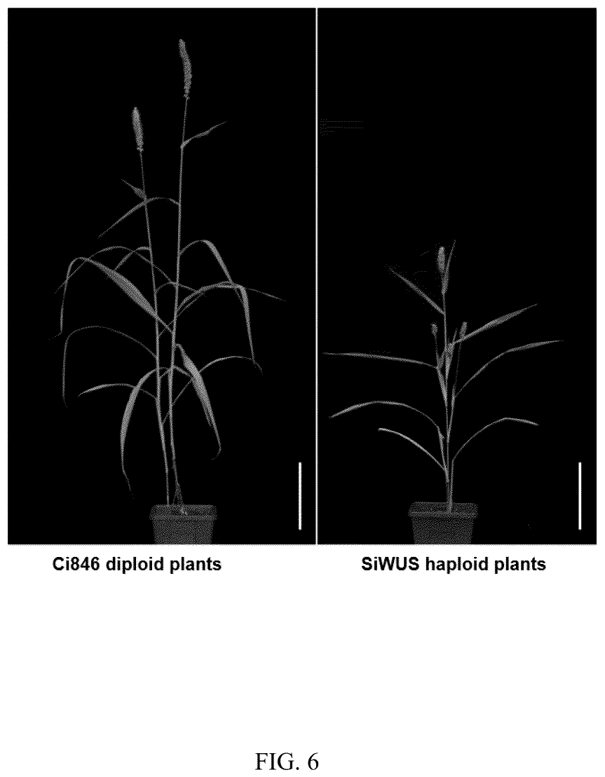

The SiWUS ectopic expression vector (pAtDD45: gSiWUS) was introduced into Ci846 following the method in Example 1. The ploidy levels of the T1 progeny were analyzed using flow cytometry. It was found that the SiWUS ectopic expression lines produced haploids at a rate of 1.1% to 2.3%. The phenotypes of SiWUS haploid plants and Ci846 diploid plants are shown in FIG. 6.

What is claimed is:

1. Method for preparing apomictic lines using a WUS gene, the method comprising the step of: introducing a WUS gene into a plant, the plant being a monocotyledon or dicotyledon from the families Poaceae, Fabaceae, or Brassicaceae, and more specifically being rice, maize, millet, wheat, barley, sorghum, soybean, or rapeseed.

2. Binary expression vector for inducing apomictic lines, the vector comprising a WUS gene driven by an egg cell-specific promoter, the WUS gene originating from monocotyledons or dicotyledons, and more specifically from plants in the families Poaceae, Fabaceae, or Brassicaceae, and most specifically from rice, maize, millet, wheat, barley, sorghum, soybean, or rapeseed, the WUS gene comprising the full-length genomic sequence or full-length coding sequence.

3. The binary expression vector of claim 2, wherein the binary expression vector also carries components for producing MiMe, the MiMe including simultaneous mutations of PAIR1, REC8, and OSD1, and the components for producing MiMe comprising: a DNA sequence encoding PARI1 sgRNA driven by a U3 promoter, a DNA sequence encoding REC8 sgRNA driven by a U3 promoter, and a DNA sequence encoding OSD1 sgRNA driven by a U3 promoter.

4. The binary expression vector of claim 2, wherein one base vector is pCAMBIA 1300; the other vector include the pCAMBIA series (excluding pCAMBIA1300), pGreen series, pBIN series, pBI series, and pHELLSGATE series; the promoter is an egg cell-specific promoter, selected from AtDD45 (At2g21740), AtEC1.1 (At1g76750), AtEC1.3 (At2g21750), AtEC1.4 (At4g39340), AtEC1.5 (At5g64720), OsECA1 (LOC_Os03g18530), OsECA2 (LOC_Os11g06730), OsECA3 (LOC_Os12g06970).

5. The binary expression vector of claim 2, wherein the rice OsWUS gene comprises the full-length genomic sequence or the full-length coding sequence of the OsWUS gene.

6. Methods for inducing apomixis in plants, the methods comprising the steps of: introducing a binary expression vector containing MiMe-producing components and a WUS ectopic expression component driven by an egg cell-specific promoter into the target plant; screening for MiMe and WUS ectopic expression lines in the transgenic plants to obtain apomictic lines.

7. The method of claim 6, wherein the plant is a monocotyledon or dicotyledon from the families Poaceae, Fabaceae, or Brassicaceae, and more specifically being rice, maize, millet, wheat, barley, sorghum, soybean, or rapeseed.

8. The method of claim 6, wherein the introduction is achieved via gene gun or Agrobacterium-mediated transformation.

9. The method of claim 6, further comprising screening for apomictic plants with normal seed-setting rates and high clonal seed efficiency.


SEQUENCE LISTING (34 sequences)

SEQ ID NO: 1 - DNA, 1051 bp, Oryza sativa
  source 1..1051: mol_type = genomic DNA
atggatcacatgcagcagcagcagcggcagcaggtgggtggagggggaggagaggaggtggcggggaggggtggtgtgccggtgtgccggccgagcgggacgaggtggacgccgacgacggagcagatcaagatcctgcgggagctgtactacagctgcggcatcaggtcgcccaactcggagcagatccagcggatcgccgccatgctgcgccagtacggccgcatcgagggcaagaacgtcttctactggttccagaaccacaaggcccgcgagcgccagaagaagcgcctcaccacgctcgacgtcaccaccaccaccgccgccgccgccgacgccgacgccagccacctcgccgtcctctccctctcgcctacagcagctggtacgttgctgcgtcatggctaattccgatcgctgcttccctgctaagctgtaatgcgcgagccggcgccgagccgccgatcgatgcttctgcgtgtgcaggcgcgacggctccctctttcccgggcttctacgtcggcaatggcggcgccgtgcagacggatcaggccaacgtcgtcaactgggactgcaccgccatggcagccgagaaaaccttcctgcaggtatgatcacacgtactactacctcctccaggtgtgtgtgaattcaccatgcaagagcaagctaatgtgcaatgctgcaggactacatgggcgtgagcggcggcggcggcgccgccgcggcggccccgacgccgtgggcgatgacgacgacgactcgcgagcccgagacgcttccactcttcccagtcgtcggcggcggcggcgacggcgcgcatcgtcacgccggccacggcggtttcccgtccaacttccagcgctggggttctgctgctgctaccaccaacaccattacggtccagcagcatttgcagcagcacaacttttacagcagcagcagcagccagctgcacagccaggatgggccggcagcaggcacatccctggagctcactctcagctcctactactgctcatgctcaccttaccctgcagggtccatgtga

SEQ ID NO: 2 - DNA, 101 bp, Oryza sativa
  source 1..101: mol_type = genomic DNA
gtacgttgctgcgtcatggctaattccgatcgctgcttccctgctaagctgtaatgcgcgagccggcgccgagccgccgatcgatgcttctgcgtgtgcag

SEQ ID NO: 3 - DNA, 80 bp, Oryza sativa
  source 1..80: mol_type = genomic DNA
gtatgatcacacgtactactacctcctccaggtgtgtgtgaattcaccatgcaagagcaagctaatgtgcaatgctgcag

SEQ ID NO: 4 - DNA, 36 bp, Oryza sativa
  source 1..36: mol_type = genomic DNA
atggatcacatgcagcagcagcagcggcagcaggtg

SEQ ID NO: 5 - DNA, 34 bp, Oryza sativa
  source 1..34: mol_type = genomic DNA
tcacatggaccctgcagggtaaggtgagcatgag

SEQ ID NO: 6 - DNA, 22 bp, Oryza sativa
  source 1..22: mol_type = genomic DNA
aagcaacccagtgcaccgctgg

SEQ ID NO: 7 - DNA, 22 bp, Oryza sativa
  source 1..22: mol_type = genomic DNA
cggagagccttagtgccatggg

SEQ ID NO: 8 - DNA, 21 bp, Oryza sativa
  source 1..21: mol_type = genomic DNA
ctgccgccgacgagcaacaag

SEQ ID NO: 9 - DNA, 45 bp, Oryza sativa
  source 1..45: mol_type = genomic DNA
ctgtcaaacactgatagtttaaatgttcctcgctgacgtaagaag

SEQ ID NO: 10 - DNA, 24 bp, Oryza sativa
  source 1..24: mol_type = genomic DNA
tattctttctttttggggtttttg

SEQ ID NO: 11 - DNA, 38 bp, Oryza sativa
  source 1..38: mol_type = genomic DNA
aaccccaaaaagaaagaataatggatcacatgcagcag

SEQ ID NO: 12 - DNA, 51 bp, Oryza sativa
  source 1..51: mol_type = genomic DNA
cctgcaggtcgactctagaggatcctcacatggaccctgcagggtaaggtg

SEQ ID NO: 13 - DNA, 25 bp, Oryza sativa
  source 1..25: mol_type = genomic DNA
ctctagagtcgacctgcaggcatgc

SEQ ID NO: 14 - DNA, 44 bp, Oryza sativa
  source 1..44: mol_type = genomic DNA
tcgtttcccgccttcagttttcccagtcacgacgttgtaaaacg

SEQ ID NO: 15 - DNA, 25 bp, Oryza sativa
  source 1..25: mol_type = genomic DNA
aggagcgctactgattcaacatgcc

SEQ ID NO: 16 - DNA, 24 bp, Oryza sativa
  source 1..24: mol_type = genomic DNA
ttctggaaccagtagaagacgttc

SEQ ID NO: 17 - DNA, 42 bp, Oryza sativa
  source 1..42: mol_type = genomic DNA
ggagtgagtacggtgtgccttcttgcgcgcgagaagagtctc

SEQ ID NO: 18 - DNA, 40 bp, Oryza sativa
  source 1..40: mol_type = genomic DNA
gagttggatgctgagtgggagatgtagtgcgtgggtcttg

SEQ ID NO: 19 - DNA, 36 bp, Oryza sativa
  source 1..36: mol_type = genomic DNA
ggagtgagtacggtgtgcttgggttagtgaggagat

SEQ ID NO: 20 - DNA, 39 bp, Oryza sativa
  source 1..39: mol_type = genomic DNA
gagttggatgctgagtggtgcgatcggaactatggagac

SEQ ID NO: 21 - DNA, 40 bp, Oryza sativa
  source 1..40: mol_type = genomic DNA
ggagtgagtacggtgtgctatcaggaggacgacgtcgccg

SEQ ID NO: 22 - DNA, 38 bp, Oryza sativa
  source 1..38: mol_type = genomic DNA
gagttggatgctgagtggctcctcctcttgggtgtagc

SEQ ID NO: 23 - DNA, 26 bp, Oryza sativa
  source 1..26: mol_type = genomic DNA
atggcggccaatgcgggcggcggtgg

SEQ ID NO: 24 - DNA, 26 bp, Oryza sativa
  source 1..26: mol_type = genomic DNA
tcacatgctccctgcagcagggtaag

SEQ ID NO: 25 - DNA, 45 bp, Oryza sativa
  source 1..45: mol_type = genomic DNA
tacgaattcgagctcggtacaaatgttcctcgctgacgtaagaag

SEQ ID NO: 26 - DNA, 24 bp, Oryza sativa
  source 1..24: mol_type = genomic DNA
tattctttctttttggggtttttg

SEQ ID NO: 27 - DNA, 46 bp, Oryza sativa
  source 1..46: mol_type = genomic DNA
aaccccaaaaagaaagaataatggcggccaatgcgggcggcggtgg

SEQ ID NO: 28 - DNA, 42 bp, Oryza sativa
  source 1..42: mol_type = genomic DNA
caggtcgactctagaggatctcacatgctccctgcagcaggg

SEQ ID NO: 29 - DNA, 24 bp, Oryza sativa
  source 1..24: mol_type = genomic DNA
atggcggccaatgtgggcggaaag

SEQ ID NO: 30 - DNA, 24 bp, Oryza sativa
  source 1..24: mol_type = genomic DNA
tcacatggtccctgcagggtaagg

SEQ ID NO: 31 - DNA, 45 bp, Oryza sativa; identical to SEQ ID NO: 25
  source 1..45: mol_type = genomic DNA

SEQ ID NO: 32 - DNA, 24 bp, Oryza sativa
  source 1..24: mol_type = genomic DNA
tattctttctttttggggtttttg

SEQ ID NO: 33 - DNA, 44 bp, Oryza sativa
  source 1..44: mol_type = genomic DNA
aaccccaaaaagaaagaataatggcggccaatgtgggcggaaag

SEQ ID NO: 34 - DNA, 44 bp, Oryza sativa
  source 1..44: mol_type = genomic DNA
caggtcgactctagaggatctcacatggtccctgcagggtaagg